(19) United States
(12) Patent Application Publication
(10) Pub. No.: US 2005/0240084 A1
(43) Pub. Date: Oct. 27, 2005

(54) System

(76) Inventors: Alyn Morice, East Yorkshire (GB); Peter Dettmar, Patrington (GB)

Correspondence Address: MYERS BIGEL SIBLEY & SAJOVEC, PO BOX 37428, RALEIGH, NC, 27627, US

(21) Appl. No.: 10/943,512

(22) Filed: Sep. 17, 2004

(30) Foreign Application Priority Data
Apr. 21, 2004 (GB) 0408836.5

(51) Int. Cl. A61B 5/00; A61B 10/00; G06F 17/00

(52) U.S. Cl. 600/300; 128/920

(57) ABSTRACT

Diagnosing a medical condition of a patient includes receiving a plurality of pieces of information on a patient medical condition, assigning weighting factors to the pieces of information, and responsive to the assigned weighting factors, determining the patient medical condition. A system for diagnosing a patient medical condition includes an interface for receiving pieces of information on a patient medical condition, means for assigning weighting factors to the pieces of information, and means for determining the medical condition of the patient responsive to assigned weighting factors. An apparatus includes a data store for storing information relating to possible conditions, a processor that receives pieces of information relating to answers to questions posed to patients, a weighting assignor that assigns weighting factors to pieces of information, and a condition determining means that determines a patient medical condition responsive to the assigned weighting factors.

This invention relates to a system and a method for diagnosing a medical condition of a patient. In particular, but not exclusively, the present invention provides a system which can allocate weightings to patients ailments or other information relating to a medical condition.

Correctly diagnosing the medical condition of a patient is crucial to enable an effective treatment for the medical condition to be achieved. For example, if the medical condition known as “chronic reflux cough”, which is caused by gastric reflux from the stomach, is incorrectly diagnosed and as a result is treated with an unsuitable medicament, such as a normal cough medicine, no beneficial therapeutic effect will be achieved. However, if the chronic reflux cough is correctly diagnosed and as a result is treated with a suitable medicament which prevents the gastric reflux, such as the medicament available under the trade name “Gaviscon”, a beneficial therapeutic effect will be achieved.

This invention is therefore aimed at providing a system and a method which enable a medical condition of a patient to be correctly diagnosed.

### STATEMENTS OF INVENTION

According to a first aspect of the present invention there is provided a method of diagnosing a medical condition of a patient, the method comprising the steps of:—
receiving a plurality of pieces of information on the medical condition of the patient;
assigning weighting factors to at least some of the pieces of information; and
responsive to the assigned weighting factors, determining the medical condition of the patient.

According to a second aspect of the present invention there is provided a system for diagnosing a medical condition of a patient, the system comprising:—
an interface for receiving a plurality of pieces of information on a medical condition of a patient;
means to assign weighting factors to at least some of the pieces of information; and
means to determine the medical condition of the patient responsive to the assigned weighting factors.

According to a third aspect of the present invention there is provided apparatus for diagnosing a medical condition of a patient comprising:—
a data store for storing information relating to a plurality of possible conditions;
a processor arranged to receive a plurality of pieces of information relating to answers to respective questions posed to said patient;
a weighting assignor for assigning weighting factors to at least some of the pieces of information; and
a condition determining means for determining a medical condition of the patient responsive to the assigned weighting factors.

By assigning carefully selected weighting factors to the information on the medical condition, the system and method according to the invention enable the medical condition of the patient to be correctly diagnosed. In particular, the system and method of the invention ensure that the appropriate degree of importance is assigned to individual pieces of information on the medical condition which are received. The medical condition is then determined taking into consideration these degrees of importance. This contrasts with the practice of general medical practitioners who generally diagnose medical conditions without assigning degrees of importance to pieces of information received in the structured manner of the invention in suit. As a result the accuracy of diagnosis using the system and method according to the invention is improved in comparison to the practice of general medical practitioners.

The system and method according to the invention enable the medical condition to be diagnosed by the patient without any direct involvement of a medical practitioner. In this manner, the invention in suit facilitates an accurate self-diagnosis of the medical condition.

In addition embodiments of the present invention allow a more comprehensive history taking to take place. This can provide a more accurate assessment of likely illnesses.

### BRIEF DESCRIPTION OF THE DRAWINGS

The invention will be more clearly understood from the following description of an embodiment thereof, given by way of example only, with reference to the accompanying drawing, in which:

FIG. 1 is a schematic illustration of a system according to the invention for diagnosing a medical condition of a patient; and

FIG. 2 illustrates a web-based system.

### DETAILED DESCRIPTION

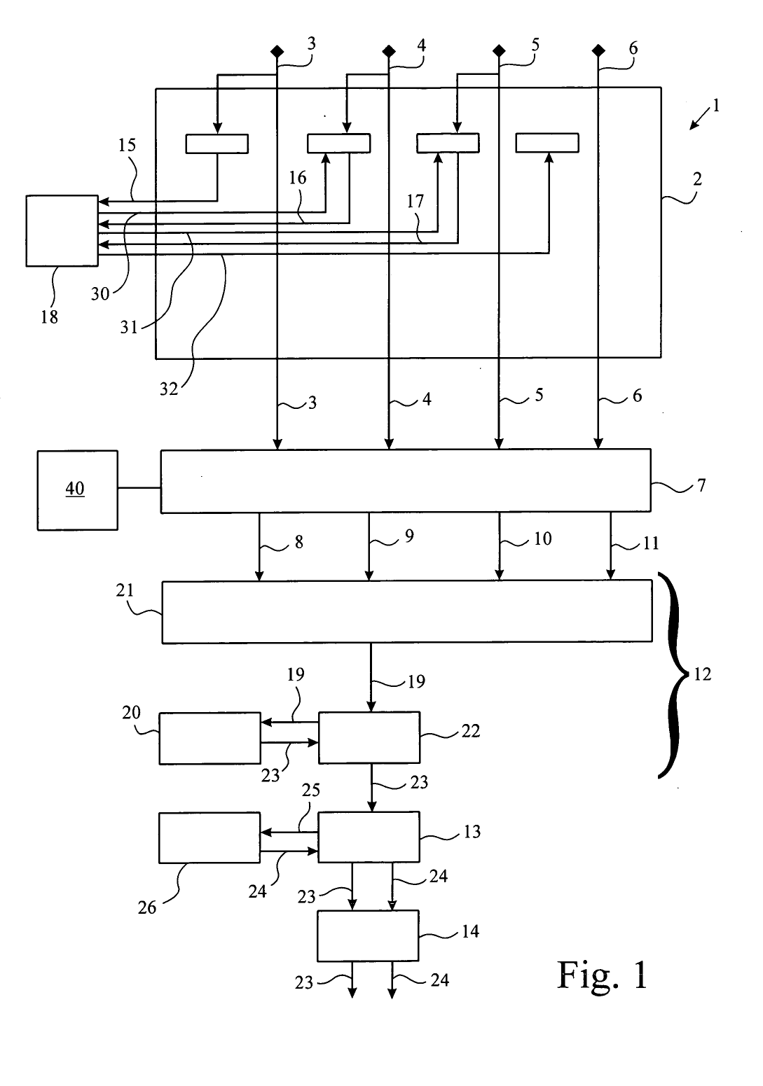

Referring to FIG. 1 there is illustrated a system, in this case a computer system 1, according to the invention for diagnosing a medical condition of a patient. The computer system 1 comprises an input interface 2 for receiving a plurality of pieces of information 3, 4, 5, 6 on the medical condition of the patient, means 7 to assign weighting factors 8, 9, 10, 11 to the pieces of information 3, 4, 5, 6, means 12 to determine the medical condition 23 of the patient, means 13 to determine a suitable treatment 24 for the medical condition 23 of the patient, and an output interface 14.

The input interface 2 displays a plurality of, and in this case four, questions on the medical condition of the patient. The pieces of information 3, 4, 5, 6 received by the input interface 2 are answers supplied by the patient or on behalf of the patient to the displayed questions. Each piece of information 3, 4, 5, 6 is sequentially received by the input interface 2 to answer the questions one after another. It will be understood that the present invention is not limited to the use of four questions. Rather any appropriate number of questions may be asked.

The input interface 2 comprises means to assign first indicators 15, 16, 17 to the answers 3, 4, 5 received, and means to then select subsequent questions from a predetermined table 18 of questions. In this manner, the input interface 2 is interactive and determines a subsequent question to display responsive to an answer received to an earlier displayed question.

The questions are expertly devised by an expert in the particular medical condition in question. The questions may take the form of a choice of possible answers, for example, often leading sequentially to the next question depending on the previous answer. For example, the questions may relate to a particular symptom of the medical condition with the patient scoring the symptom depending on severity, for example, on the basis of a 1 to 10 score. Alternatively, the questions may involve a numerical reply from the patient such as date of birth, number of cigarettes smoked per day etc.

Examples of some possible questions in the predetermined table 18 of questions are:
How long have you had a cough?
Severity of cough: Indicate on a scale how severe you feel your cough is. 0 is no cough at all and 10 is a continuous most disturbing cough (unbearable symptoms).
Was cough preceded by chest/upper airway infection?
Do you use any inhalers?
   Do you take a steroid inhaler?
   Do you take a reliever inhaler?
   Do you take a long acting reliever?
   Do you take an ipratropium inhaler?
   Do you take a combined reliever inhaler?
   Do you take a combined steroid inhaler?
Do you take steroid tablets?
   Do you take them: every day? intermittently?
Do you take antihistamine tablets?
Do you take medication for blood pressure or for your heart?
   Do you take a beta-blocker?
   Do you take an ACE inhibitor?
Have you been diagnosed with any breathing problems other than chronic cough?
If yes, tick the appropriate conditions:
   Asthma
   Emphysema
   Bronchiectasis
   COPD
Please tick the box
   (a) I have never smoked.
   (b) I smoke at the moment.
      If yes, how many cigarettes a day?
      For how many years?
   (c) I don't smoke now but have in the past.
      If yes, how many cigarettes a day?
      For how many years?
      How long ago did you stop smoking?
Please give a rating for how the following problems affect you (0=no problem, 5=severe problem).
Hoarseness or a problem with your voice.
Clearing your throat.
Excess mucus in the throat, or drip down the back of your nose.
Difficulty swallowing food, liquids or pills.
Coughing after lying down.
Breathing difficulties or choking episodes.
Troublesome or annoying cough.
Sensations of something sticking in your throat, or a lump in your throat.
Heartburn, chest pain, indigestion or stomach acid coming up.
Cough with eating (during or straight after meals).
Cough with certain foods.
Cough waking you from sleep.
Cough when you get up out of bed in the morning.
Cough brought on by singing or by speaking (for example, on the telephone).
Wheezing or chest tightness in general.
Wheezing or chest tightness which is worse in the early mornings or which wakes you from sleep.
Wheezing or breathlessness brought on by dust, pollen or animals.
Wheezing or breathlessness brought on by exercise.
Worsening of wheeze or breathlessness after use of aspirin or beta-blockers (such as atenolol).
Coughing up phlegm or sputum which is coloured (green or yellow).
Coughing up more than 1 cupful of phlegm or sputum in a day.
Coughing up blood.
Blocked or stuffy nose.
Runny nose.
Itchy nose and/or sneezing.
Loss of the sense of smell.
Sensation of pain or pressure in your face (around the nose or eyes).

Each answer 3, 4, 5, 6 is assigned a weighting factor 8, 9, 10, 11 respectively by the weighting factor means 7. The weighting factors are chosen on the basis of patient responses given in a number of surveys carried out prior to initialisation of the system. In addition surveys may continually be carried out and weighting factors adjusted. Patients with a known diagnosis such as reflux cough fill in questionnaires which permit the compilation of a weighting scheme. A table of responses is then constructed allowing the individual weighting of questions.

The medical condition determination means 12 comprises means 21 to determine a second indicator 19, and means 22 to select the medical condition 23 of the patient from a predetermined table 20 of medical conditions.

The second indicator determination means 21 determines the second indicator 19 by as will be described hereinafter.

The treatment determination means 13 comprises means to assign a third indicator 25 to the medical condition 23 of the patient, and means to select a suitable treatment 24 from a predetermined table 26 of treatments.

The output interface 14 may display the medical condition 23 of the patient and/or the suitable treatment 24. Additionally or alternatively the output interface 14 may print out the medical condition 23 of the patient and/or the suitable treatment 24.

A computer program product is provided having computer program code to control operation of the computer system 1.

A skilled addressee will appreciate that the input interface 2 and the output interface 14 may be provided in the form of a single piece of hardware, or in the form of separate pieces of hardware.

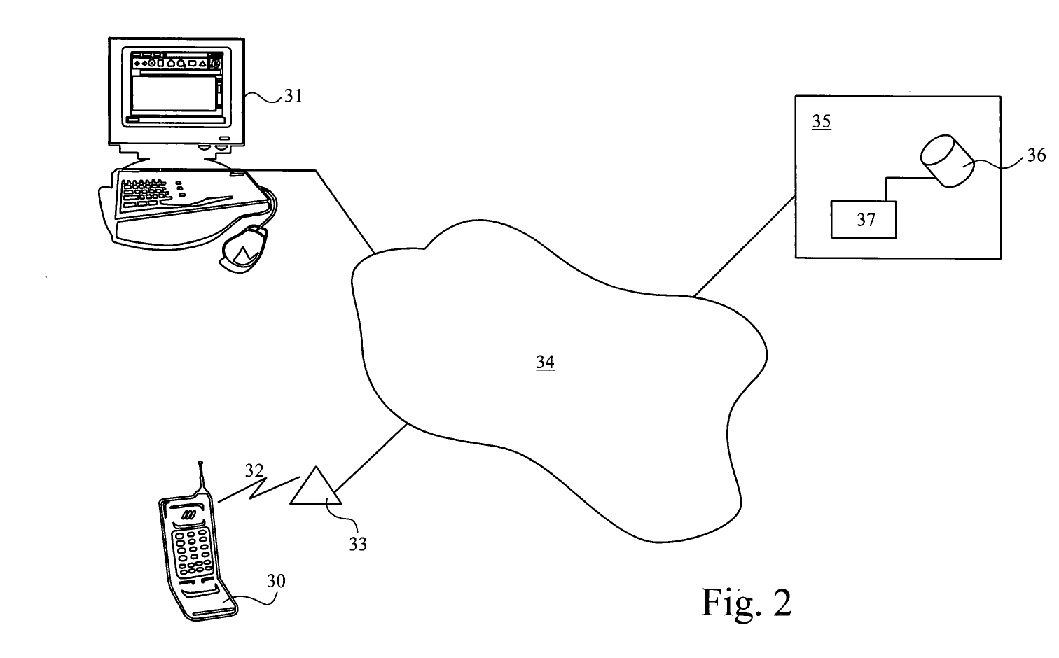

Furthermore, the input interface 2 and/or the output interface 14 may be configured to enable the patient to interact with the system 1 from a remote location. In one case, the system 1 may be accessible to a remote patient by means of an internet and/or intranet connection. FIG. 2 illustrates a web-based diagnosis system in accordance with an embodiment of the present invention. A user who may be a patient or a guardian of a patient logs on to a predetermined website via a user terminal. The user terminal may be a remote terminal such as mobile phone 30 or PC 31. The mobile phone is connected via a wireless link 32 to a base station 33 and then via fixed connection to the internet 34. A host node 35 includes a database 36 for storing the various tables and a processor 37 for processing data.

The predetermined tables 18, 20, 26 may be provided in any suitable form, such as in the form of a database.

In use, the input interface 2 initially displays a first question on the medical condition of the patient. This is displayed on a user display on a respective user terminal 31, 30. The patient or a person on behalf of the patient supplies an answer 3 to this first displayed question. The answer 3 is received by the input interface 2 which assigns an indicator 15 to the answer 3. This indicator 15 is then routed to the predetermined table 18 of questions, where a second question 30 is selected responsive to the indicator 15. The second question 30 on the medical condition of the patient is routed back to the input interface 2 and displayed.

Thus, the input interface 2 is interactive. The second question 30 is selected in response to the answer 3 to the first question.

When an answer 4 to the second question 30 is received by the input interface 2, the input interface 2 assigns an indicator 16 to the answer 4. This indicator 16 is then routed to the predetermined table 18 of questions, where a third question 31 is selected responsive to the indicator 16. The third question 31 on the medical condition of the patient is routed back to the input interface 2 and displayed.

When an answer 5 to the third question 31 is received by the input interface 2, the input interface 2 assigns an indicator 17 to the answer 5. This indicator 17 is then routed to the predetermined table 18 of questions, where a fourth and final question 32 is selected responsive to the indicator 17. The fourth question 32 on the medical condition of the patient is routed back to the input interface 2 and displayed.

When an answer 6 to the fourth question 32 is received by the input interface 2, all of the answers 3, 4, 5, 6 are then routed to the weighting factor means 7, where weighting factors 8, 9, 10, 11 are assigned to each of the answers 3, 4, 5, 6 respectively.

The weighting factor is determined in accordance with a weighting factors table 40 stored in the system. This data store contains for each question and for each condition a weighted value for each possible response. These weighting values are predetermined according to information known about the condition and other factors. For each response to the questions 3, 4, 5, 6 and any questions in table 18 a table 40 is referenced. The table contains an entry for each defined condition in table 20 and each possible response to questions 3, 4, 5, 6 and table 18. By applying these values, weighted values 8, 9, 10, 11 are established for individual answers.

Responsive to the four weighting factors 8, 9, 10, 11, the second indicator 19 is then determined in the second indicator determination means 21. The sum of weighted values is then taken for each defined condition. For each defined condition this factor is converted to a percentage of the maximum possible score for that condition. Indicator 19 is the final calculated percentage score for each defined condition. When this process is complete the most likely condition 23 is selected as that with the highest percentage value in indicator 19.

This second indicator 19 is routed to the table 20 of medical conditions, where the medical condition 23 of the patient is selected responsive to the second indicator 19.

The medical condition 23 of the patient is routed on to the treatment determination means 13, where a third indicator 25 is assigned to the medical condition 23. This third indicator 25 is routed to the table 26 of treatments, where the suitable treatment 24 for the medical condition 23 is selected responsive to the third indicator 25. Treatment 24 is identified by referencing condition 23 in table 26. As an example of these overriding factors for example treatment for diabetes may differ from treatment to non-diabetics. Thus table 26 includes a variety of treatments for the defined conditions subject to predefined factors established through the input interface 2.

Finally, the medical condition 23 of the patient and the suitable treatment 24 are routed to the output interface 14, where the medical condition 23 and/or the suitable treatment may be displayed and/or printed out. The system 1 thus provides a simple and fast means for a patient to obtain an accurate diagnosis and a recommended treatment without direct contact with a medical practitioner being required. In this way the system 1 enables the patient to perform an accurate self-diagnosis.

In certain cases, the recommended treatments for particular medical conditions may vary from country to country. To ensure that the system and method according to the invention may be used irrespective of the geographical location of the patient, the system may comprise means to determine the geographical location of the patient and may incorporate a plurality of tables of treatments for different countries. When the system determines the geographical location of the patient, the system selects the particular table of treatments which is appropriate for the country of the patient. It is this particular table of treatments which is then accessed with the third indicator 25 to select the suitable treatment 24 for the medical condition 23 of the patient. In this manner, the treatment 24 recommended to the patient is in accordance with the medical guidelines of the patient's country. The patients country may be derived from a number of different techniques for example by simply asking the patient and/or patients guardian to input country information or when the system is accessed remotely via the internet by extracting a country indicating data from the unique IP address for that specific computer or mobile terminal being used by a user. Using the first of these options allows a person to register with the diagnostic system whilst outside their native country.

A second questionnaire may be sent to the patient at a set period after completing the original questionnaire and having received a diagnosis of their symptoms (for example, within 4-8 weeks). The purpose of the second questionnaire will be to ask the patient to complete questions on how they found the medicine they were prescribed and how their symptoms responded to the medication. The results from the second questionnaire will be used to ascertain the acceptability and efficacy of the medication taken which will result in a web-based method of collecting clinical trial data.

Although the system 1 has been described above with only four pieces of information 3, 4, 5, 6 being received by the input interface 2, it will be appreciated that any suitable number of pieces of information on the medical condition of the patient may be received.

It will further be appreciated that although the invention has been described in relation to the diagnosis of a cough, the system and method according to the invention are also suitable for diagnosing a variety of other medical conditions, such as heartburn, asthma, hypertension, osteoporosis. The invention is particularly suitable for diagnosing medical conditions for which national and/or international guidelines relating to diagnosis and treatment are available. Embodiments of the present invention can be used to diagnose many various different types of medical conditions such as asthma, chronic obstructive pulmonary disease, hypertension, heart failure, diabetes and symptoms such as heartburn, cystitis and/or chronic pain.

Embodiments of the present invention also enable people in countries short of high quality medical facilities or doctors to access first rate medical knowledge.

Embodiments of the present invention thus provide a method of self-diagnosis which most advantageously may be carried out by the patient. The method relates particularly, though not exclusively, to common ailments such as coughs and chronic coughs, heartburn, asthma, hypertension and osteoporosis. Also the methods are particularly, though not exclusively, applicable to those ailments for which national and/or international guidelines relating to diagnosis and treatment are available.

Embodiments of the present invention provide a web-based method that has the following sequence of steps:

Step 1—a pro-form a series of questions relating to a particular ailment selected by the patient is filled out by the patient. The questions are expertly devised to generate a sequence of information which is translated into a score. The questions are devised by an expert in the particular ailment in question. The questions take the form of a choice of possible answers, for example, often leading sequentially to the next question depending on the previous answer. For example, a question may relate to a particular symptom of the ailment for the patient scoring the symptom depending on severity, for example, on the basis of a 1:10 score. Alternatively, the questions may involve a numerical reply from the patient such as date of birth, number of cigarettes smoked per day, etc. All the questions are set so that the answers can be attributed a score and a total score may be calculated.

Step 2—relates to the generation of a score per answer resulting in a final score for the patient in question. Hence, a database is devised including all possible answers for the questions set, scoring each answer so that a permutation of answers will result in a final score to result in a correct diagnosis for the patient.

Step 3—relates to the final score achieved to a database that provides a diagnosis for the patient's condition within the particular ailment selected. For example, if the ailment selected is a cough, the final score may point to the patient having, for example, a reflux cough.

Step 4—relates to the diagnosis to the national and/or international guidelines for the ailment selected. The diagnosis is matched to the recommended advice provided in the guidelines appropriate for the ailment. The patient is provided with the recommended course of action, such as treatment, for the diagnosis made. For example, for the diagnosis of a reflux cough, the national guidelines for coughs recommend that Gaviscon™ is used to treat a reflux cough. Hence, the end result of the patient will be a recommendation that Gaviscon is taken as per the guidelines on the bottle.

Hence embodiments of the present invention provide a way in which the patient can apply the national and/or international guidelines to their own particular problem without recourse to a medical expert and avoiding the need to plough through lengthy national guidelines relating to a particular ailment which may be baffling to a patient and may not provide the patient with a meaningful outcome relating to their condition.

Since embodiments of the present invention provide a web-based method, the program also enables patient to obtain advice from the national/international guidelines of the country from where they are dialling into the website. Hence the program is able to nationalise the advice given, particularly in relation to the type of medication required.

Embodiments of the present invention also relate to patients receiving a second follow-up questionnaire at a set period after completing the original questionnaire or after some predetermined event has occurred and having received a diagnosis of their symptoms (for example, within 4-8 weeks). The purpose of the second questionnaire is to ask patients to complete questions on how they found the medicine they were prescribed and how their symptoms responded to the medication. The results from the second questionnaire may be used to ascertain the acceptability and efficacy of the medication which will result in a web-based method of collecting clinical trial data.

The invention is not limited to the embodiment hereinbefore described, with reference to the accompanying drawing, which may be varied in construction and detail.

1. A method of diagnosing a medical condition of a patient, the method comprising the steps of:
receiving a plurality of pieces of information on the medical condition of the patient;
assigning weighting factors to at least some of the pieces of information; and
responsive to the assigned weighting factors, determining the medical condition of the patient.

2. A method as claimed in claim 1 further comprising:
providing the patient with a follow-up questionnaire subsequent to said step of determining the medical condition.

3. A method as claimed in claim 2 further comprising:
providing the patient with treatment for the determined medical condition and providing the follow-up questionnaire subsequent to the provision of said treatment.

4. A method as claimed in claim 1 wherein the plurality of pieces of information are received sequentially.

5. A method as claimed in claim 1 wherein the method comprises the step of displaying one or more questions on the medical condition of the patient.

6. A method as claimed in claim 5 wherein the piece of information received comprises an answer to a displayed question.

7. A method as claimed in claim 5 or 6 wherein the method comprises the steps of:
displaying a plurality of questions on the medical condition of the patient; and
determining a subsequent question to display responsive to an answer received to an earlier displayed question.

8. A method as claimed in claim 7 wherein the subsequent question is determined by:
assigning a first indicator to the answer received to the earlier displayed question; and
responsive to the assigned first indicator, selecting the subsequent question from a predetermined table of questions.

9. A method as claimed in claim 1 wherein the method comprises the step of:
responsive to the assigned weighting factors, determining a second indicator;
the medical condition of the patient being determined responsive to the second indicator.

10. A method as claimed in claim 9 wherein the second indicator is determined by the steps of:
determining a final calculated percentage score for each defined condition.

11. A method as claimed in claim 9 wherein the medical condition of the patient is determined by, responsive to second indicator, selecting the medical condition of the patient from a predetermined table of medical conditions.

12. A method as claimed in claim 1 wherein the method comprises the step of determining a suitable treatment for the medical condition of the patient based on Regional, National and/or International guidelines.

13. A method as claimed in claim 12 wherein the suitable treatment is determined by:
assigning a third indicator to the medical condition of the patient; and
responsive to the assigned third indicator, selecting the suitable treatment from a predetermined table of treatments.

14. A method as claimed in claim 13 wherein the method comprises the steps of:
determining the geographical location of the patient; and
responsive to the geographical location of the patient, selecting an appropriate table of treatments from a plurality of tables of treatments.

15. A method as claimed in claim 1 wherein the method comprises the step of displaying the medical condition of the patient and/or the suitable treatment.

16. A method as claimed in claim 1 wherein the method comprises the step of printing out the medical condition of the patient and/or the suitable treatment.

17. (canceled)

18. A computer program product comprising computer program code capable of causing a computer system to perform a method as claimed in claim 1 when the computer program product is run on a computer system.

19. A system for diagnosing a medical condition of a patient, the system comprising:
an interface for receiving a plurality of pieces of information on a medical condition of a patient;
means to assign weighting factors to at least some of the pieces of information; and
means to determine the medical condition of the patient responsive to the assigned weighting factors.

20. A system as claimed in claim 19 wherein the interface is configured to display one or more questions on a medical condition of a patient.

21. A system as claimed in claim 20 wherein the interface is configured to display a plurality of questions on a medical condition of a patient, and the system comprises means to determine a subsequent question to display responsive to an answer received to an earlier displayed question.

22. A system as claimed in claim 21 wherein the subsequent question determination means comprises:
means to assign a first indicator to an answer received to an earlier displayed question; and
means to select a subsequent question from a predetermined table of questions responsive to the assigned first indicator.

23. A system as claimed in claim 19 wherein the system comprises means to determine a second indicator responsive to the assigned weighing factors, the medical condition determination means being configured to determine the medical condition of the patient responsive to the second indicator.

24. A system as claimed in claim 23 wherein the second indicator determination means is configured to determine the second indicator by determining a final calculated percentage score for each defined condition.

25. A system as claimed in claim 23 wherein the medical condition determination means is configured to select the medical condition of the patient from a predetermined table of medical conditions responsive to the second indicator.

26. A system as claimed in claim 19 wherein the system comprises means to determine a suitable treatment for the medical condition of the patient based on regional and/or international guidelines.

27. A system as claimed in claim 26 wherein the suitable treatment determination means comprises:
means to assign a third indicator to the medical condition of the patient; and
means to select a suitable treatment from a predetermined table of treatments responsive to the assigned third indicator.

28. A system as claimed in claim 27 wherein the system comprises:
means to determine the geographical location of a patient; and
means to select an appropriate table of treatments from a plurality of tables of treatments responsive to the geographical location of the patient.

29. A system as claimed in claim 19 wherein the interface is configured to display the medical condition of the patient and/or the suitable treatment.

30. A system as claimed in claim 19 wherein the system comprises means to print out the medical condition of the patient and/or the suitable treatment.

31. Apparatus for diagnosing a medical condition of a patient comprising:
a data store for storing information relating to a plurality of possible conditions;
a processor arranged to receive a plurality of pieces of information relating to answers to respective questions posed to said patient;
a weighting assignor for assigning weighting factors to at least some of the pieces of information; and
a condition determining means for determining a medical condition of the patient responsive to the assigned weighting factors.

32. (canceled)

33. (canceled)
